(19) United States
(12) Patent Application Publication
(10) Pub. No.: US 2017/0049395 A1
(43) Pub. Date: Feb. 23, 2017

(54) NEURO-VIGILANCE INTEGRATED CONTACT EYE LENS AND SYSTEM

(71) Applicant: Zansors LLC, Tysons, VA (US)

(72) Inventor: Hung CAO, Kenmore, WA (US)

(73) Assignee: Zansors LLC, Tysons, VA (US)

(21) Appl. No.: 15/241,941

(22) Filed: Aug. 19, 2016

Related U.S. Application Data
(60) Provisional application No. 62/207,553, filed on Aug. 20, 2015.

(51) Int. Cl. A61B 5/00; A61B 3/113; A61B 5/0205; A61B 3/11

(52) U.S. Cl. CPC A61B 5/6821; A61B 3/112; A61B 3/113; A61B 5/0205; A61B 5/02438

(57) ABSTRACT

A contact lens may comprise at least one sensor. An eye gear package may comprise an interface circuit configured to communicatively couple the eye gear package with the contact lens, a power source configured to power the eye gear package and the contact lens, and a processor configured to process data generated by the at least one sensor.

### CROSS-REFERENCE TO RELATED APPLICATIONS

This application claims priority from U.S. Provisional Application No. 62/207,553, filed Aug. 20, 2015, the entirety of which is incorporated by reference herein.

### BACKGROUND

A person's Sympathetic Nervous System (SNS) can be indirectly assessed with either immobile, wired devices or wrist devices capturing skin conductance, motion, and heart rate—yet these are not validated for SNS activities. Many of these existing tools are battery powered and limited by the battery's operation (e.g., about 6-10 hours).

### SUMMARY

Pupilometry is a physiological representation of neurological function (e.g. after stroke), physiological stresses, emotional reactions, and attention processes (e.g. that change with development or aging). Pupil diameter is under the control of the sympathetic nervous system (SNS). Systems and methods described herein may provide a non-invasive system capable of measuring high-fidelity SNS activity for the long term, low stress monitoring of animals in research facilities and in studies of animals that model human diseases, in addition to humans. An indirect, but accurate, real-time, SNS activity measurement system may include a portable, non-invasive system comprising a wearable device capable of detecting electroencephalography and a wirelessly-coupled smart contact lens capable of providing simultaneous detection of pupil diameter, light levels, and eye movements.

Embodiments disclosed herein may provide a Neuro-vigilance Integrated Contact Eye (N.I.C.E.) lens and system to measure pupil diameter and eye movement of the person wearing the lens and system.

Embodiments disclosed herein may provide a Neuro-vigilance Integrated Contact Eye (N.I.C.E.) lens in communication with an eye gear, which together may form a system for measuring, processing, and communicating, among other things, pupil diameter and eye movement of the person wearing the lens and eye gear.

Embodiments disclosed herein may also detect, monitor, process and/or communicate electroencephalogram (EEG), electrooculogram (EOG), heart rate, and skin impedance data.

### BRIEF DESCRIPTION OF THE DRAWINGS

FIGS. 1A and 1B show a N.I.C.E. system according to an embodiment of the invention.

FIG. 2 shows a N.I.C.E. system block diagram according to an embodiment of the invention.

FIG. 3 shows a tilting angle detection according to an embodiment of the invention.

FIG. 4 shows examples of eye movement speed detections according to an embodiment of the invention.

FIG. 5 shows a load modulation arrangement according to an embodiment of the invention.

FIG. 6 shows a strain detection process according to an embodiment of the invention.

FIG. 7 shows a movement detection process according to an embodiment of the invention.

FIG. 8 shows a manufacturing process according to an embodiment of the invention.

### DETAILED DESCRIPTION OF SEVERAL EMBODIMENTS

Systems and methods described herein may provide contact lens technology that may perform simultaneous detection of pupil diameter and eye movement. A N.I.C.E. (Neuro-vigilance Integrated Contact Eye) lens may measure pupil diameter and eye movement in a single contact lens. The disclosed lens may allow science and technology to move away from invasive measurements of the Sympathetic Nervous System (SNS), and instead use the N.I.C.E. lens as a non-invasive tool for SNS-related (e.g., sleep) investigations and applications. Together with a comfortable polymer-based “eye gear” providing a power supply, communications, and the detection of electroencephalogram (EEG), electrooculogram (EOG), heart rate and skin impedance data, the disclosed N.I.C.E. lens and its system may allow for more definitive detection of rapid eye movement (REM) sleep and studies of theta power changes during sleep after trauma that track post-traumatic stress disorder (PTSD) in susceptible patients, for example.

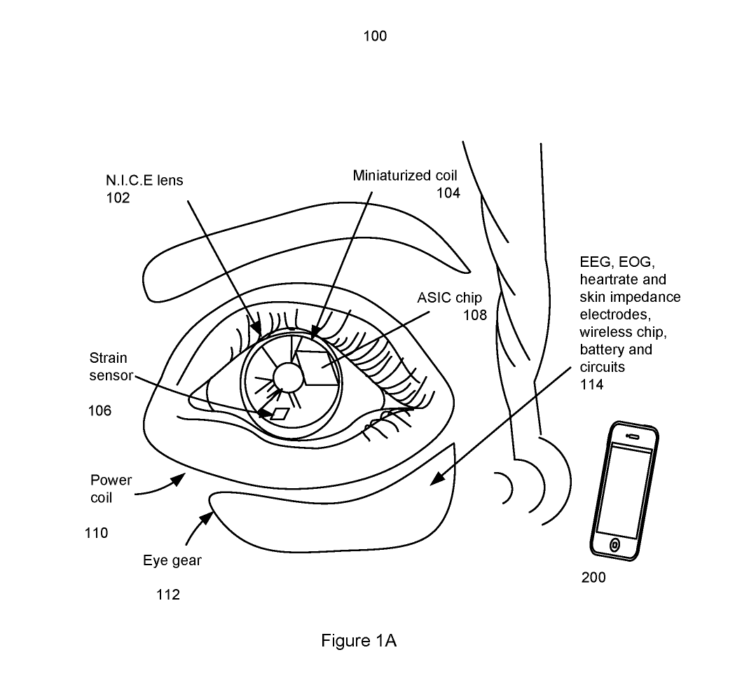

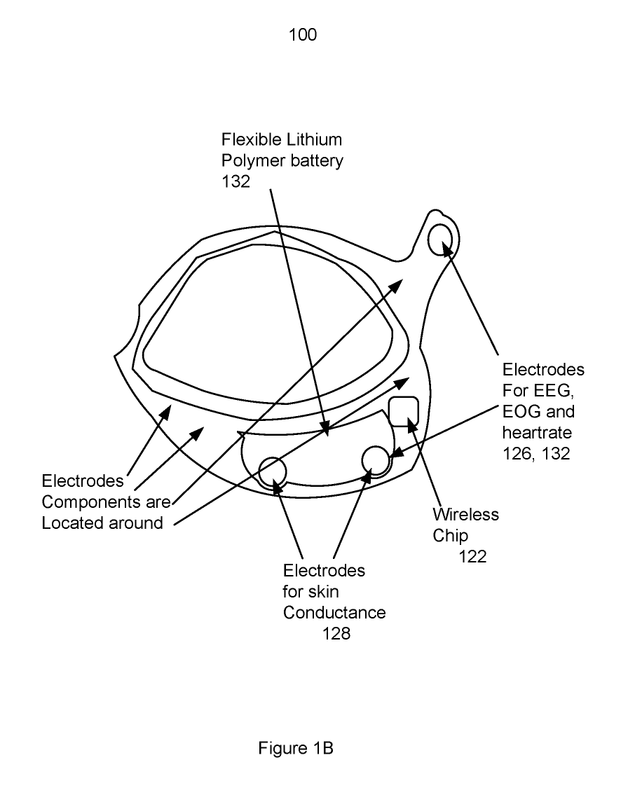

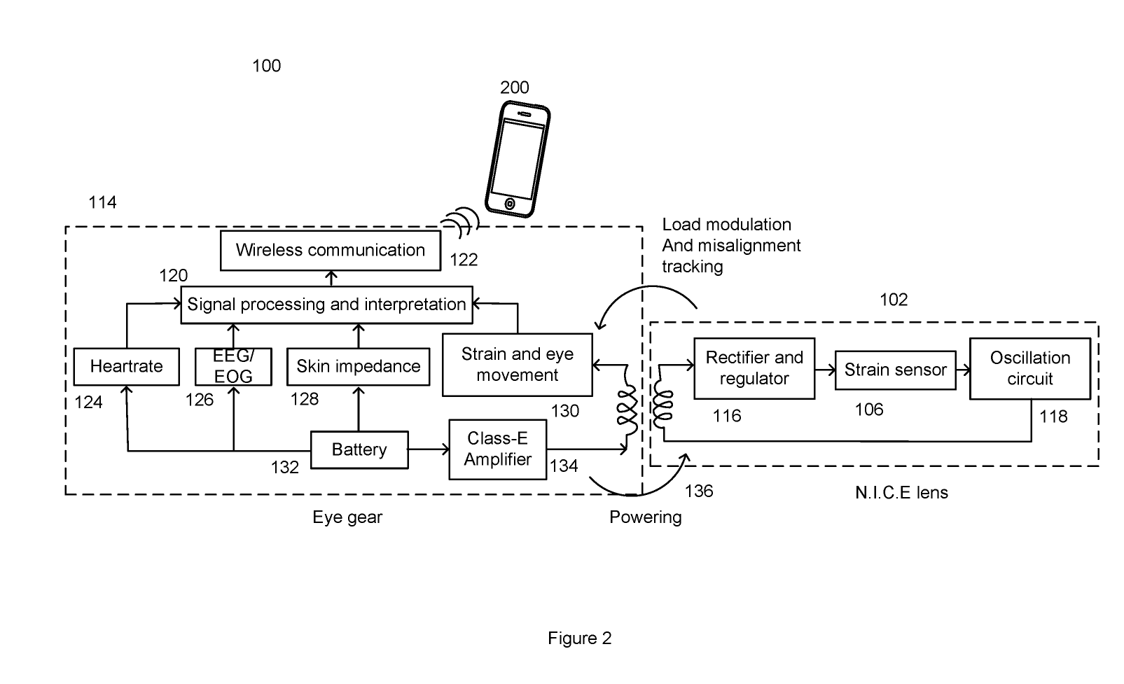

FIGS. 1A and 1B show a N.I.C.E. system 100 according to an embodiment of the invention. The system 100 may be designed based on micro-electromechanical systems (MEMS) technology developed on a thin film of contact-lens material forming the lens 102, such as polydimethylsiloxane (PDMS). In some embodiments, the system's powering mechanism may be inductive coupling between two antennas in the eye gear (power coil 110) and the N.I.C.E lens (miniaturized coil 104), respectively. The operating frequency may be chosen at the near field communication (NFC) frequency of 13.56 MHz, for example. A miniaturized coil 104 may harvest the energy sent from the power source in the eye gear 112 via inductive coupling to operate the circuit components within the lens 102, which may provide efficiency and biocompatibility. The sensory data, including pupil dilation and eye movement, may be sent from the coil 104 to the power coil 110 in the eye gear 112 via backscattered load modulation. A fabricated coil 104 can be used, and the film thickness may be increased by electroplating it to enhance its quality factor. Since the distance between the coils may be almost zero, sufficient power may be delivered. A smart MEMS-based carbon strain microsensor 106 or other strain sensor may be integrated into the lens 102. Circuit elements of the lens 102, such as those described in FIG. 2, may be arranged in an application-specific integrated circuit (ASIC) chip 108 or other circuit element. The ASIC chip 108 may be back-side etched in a reactive ion etching (RIE) chamber to obtain a thickness of 30 μm or similar to be flexible before integration, as described below. The eye gear 112 may be fabricated on a parylene C substrate, containing thin-film goal electrodes, power coil 110, and circuit routings to assemble electronics (circuitry 114). Data, such as EEG, EOG, heartrate, and skin impedance, may be gathered (e.g., through electrode and/or communication with the lens 102 as described below) and sent to a smartphone connected to the cloud integrated with a diagnostic system 200 for distanced care. The wireless communication between the eye gear 114 and a smartphone may be done via low-power Bluetooth or other suitable wireless communications, and a Lithium-polymer battery 132 may be used to keep the entire eye gear 112 flexible. The N.I.C.E. lens 102 may be capable of measuring pupil diameters indirectly via the strain sensor 106 and eye movement via the misalignment between the coils 104 in the lens and the eye gear 110, respectively, as described below.

FIG. 2 shows a N.I.C.E. system 100 block diagram according to an embodiment of the invention. The eye gear 112 circuitry may include signal processing and interpretation circuitry 120, wireless communication circuitry 122 which may be used to communicate data with the cloud or cloud-based network or diagnostic system 200, heartrate sensor 124, EEG sensor 126, skin impedance sensor 128, strain and eye movement sensor 130, battery or other power source 132, and/or an amplifier (e.g., class-E amplifier 134). The N.I.C.E. lens 102 may include a strain sensor 106 as mentioned above, an oscillation circuit 118, a rectifier and regulator 116. The amplifier 134 may provide power to the N.I.C.E. lens 102 electronics through the energy harvest via inductive coupling between coils 104 and 110, which may be rectified by the rectifier 116. The N.I.C.E. lens 102 electronics may provide feedback for load modulation and misalignment tracking to the circuitry 114 through the inductive coupling link.

In some embodiments, both the N.I.C.E. lens 102 and the eye gear 114 may be based on flexible polymer with embedded components and sensors. The circuit routings, inductor antenna, and electrodes in the eye gear 114 may be made of 0.5-um thick gold sputtered film, for example. The strain sensor may be developed by sputtering carbon onto a 10-um thick parylene C film and etched to define a final size of 0.5 mm×1 mm. Circuits in the N.I.C.E lens 102 may be integrated into an application-specific integrated circuit (ASIC) chip. The ASIC chip, at 3 mm×3 mm, may be back-side etched to obtained a thickness of 30 μm so that it becomes flexible. The thinned ASIC chip and the strain sensor may be integrated and encapsulated in the N.I.C.E lens 102 with connection to the coil 104 for powering and operation.

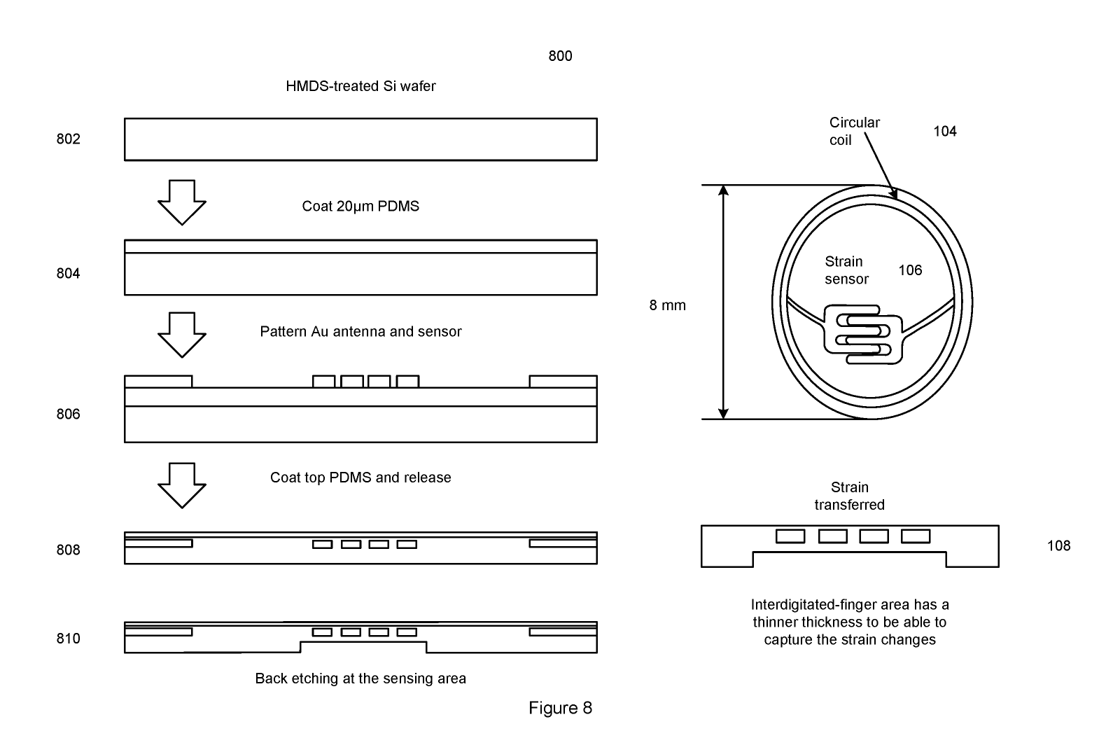

FIG. 8 shows an example process 800 for creating ASICs for both the N.I.C.E lens 102 and the eye gear 114. An hexamethyldisilazane-treated (HMDS) silicon wafer may be processed 802. First, 20 μm parylene C may be spin coated onto the wafer 804. Next, a double layer of Au (0.2 μm) on Ti (0.02 μm) may be deposited and patterned 806, followed by another deposition of 10-μm thick parylene to encapsulate the entire device 808. The device may be back-etched at the location of the carbon strain sensor to ensure the strain information from the iris muscle is effectively transferred to the sensing area 810 and then released in water. For the N.I.C.E lens 102, the entire system may be then encapsulated inside the lens material PDMS. For the eye gear 114, a gecko-inspired texture may be formed on the surface to secure it on the skin while bringing comfort to user.

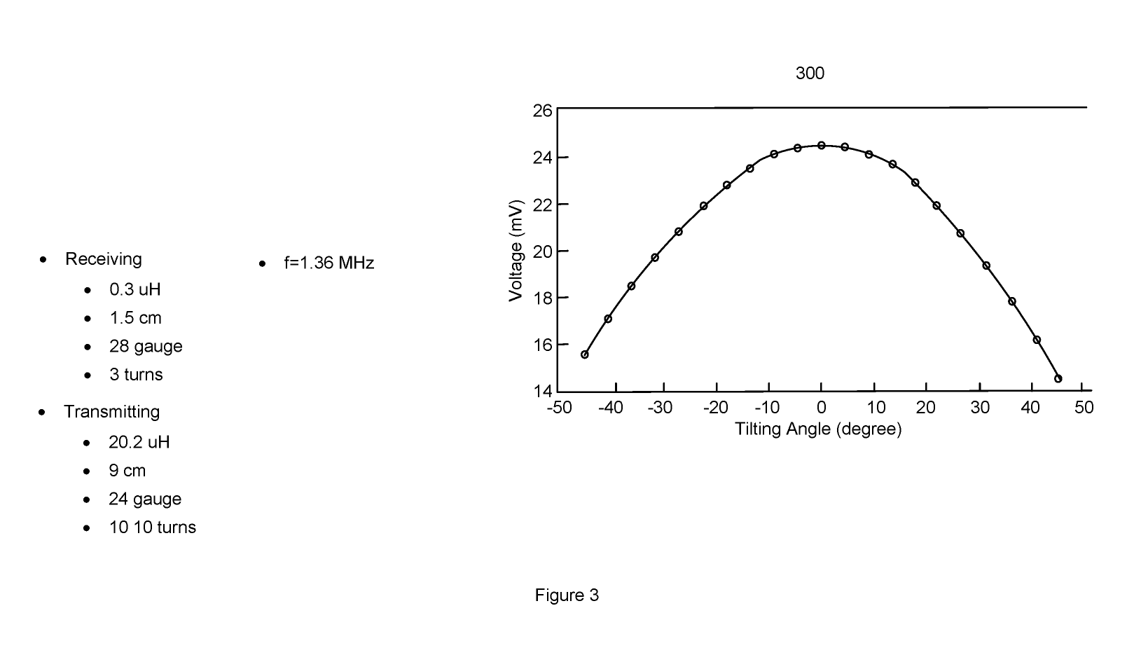

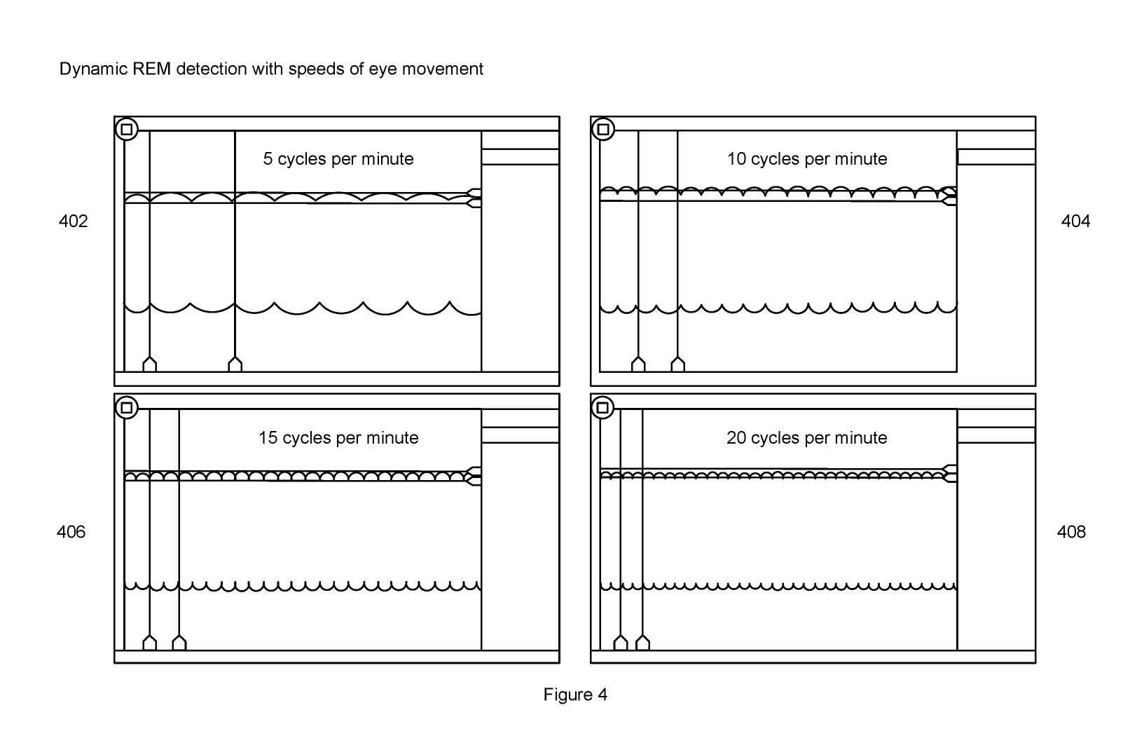

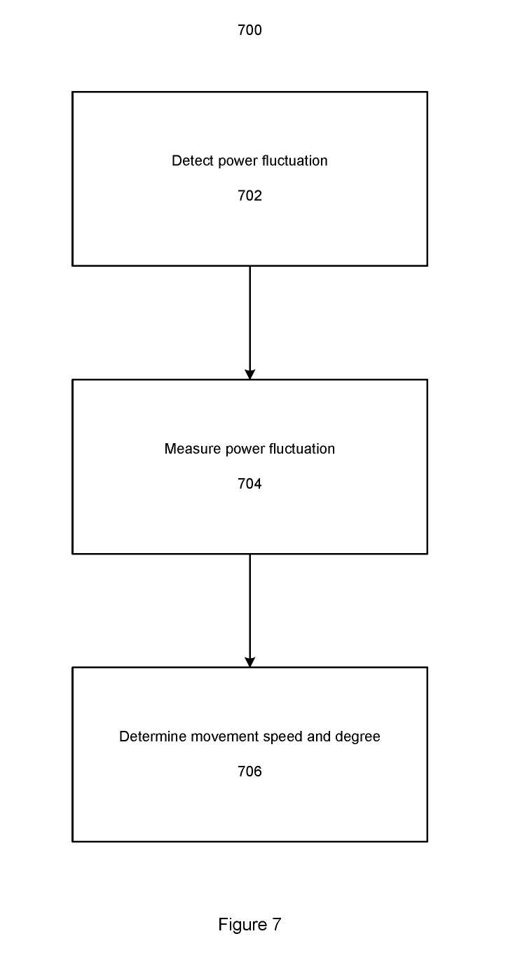

FIG. 7 shows a movement detection process 700 according to an embodiment of the invention. As noted above, the system 100 may detect eye movement and speed. As the eye ball moves, the coil 104 may become misaligned with respect to the coil 110, thus the received power will be fluctuated. in The power fluctuations may be detected 702 by strain and eye movement sensor 130 and measured 704 by signal processor 120, and based on the measured fluctuations, the speed of the movement as well as the degree of the movement may be tracked 706 by signal processor 120 and reported by wireless communication system 122. FIG. 3 shows a tilting angle detection example 300. As shown by the graph, changes in voltage represent changes in tilting angle of the eye. Furthermore, a slope of the curve represents the speed of the movement of the eye. FIG. 4 shows additional examples of REM detection made by the process 400 of FIG. 7. For example, graph 402 shows 5 cycle per minute eye movement, graph 404 shows 10 cycle per minute eye movement, graph 406 shows 15 cycle per minute eye movement, and graph 408 shows 20 cycle per minute eye movement. As shown, faster eye movement results in steeper curves on the graph.

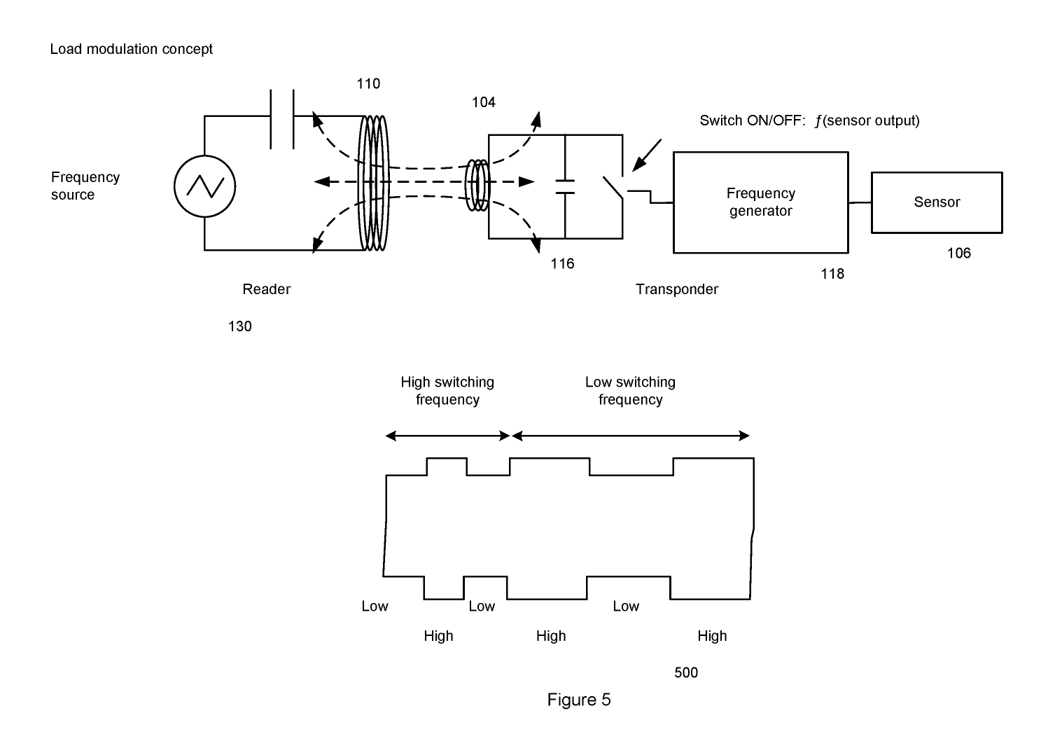

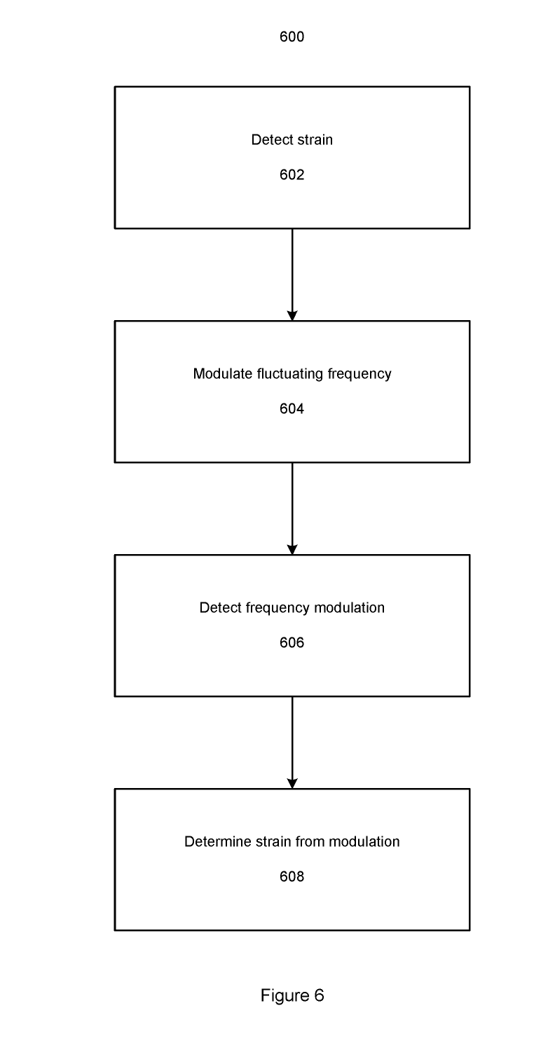

FIG. 5 shows a load modulation arrangement using elements of the circuit of FIG. 2, and FIG. 6 shows a strain detection process 600 according to an embodiment of the invention. As the iris muscle contracts, the strain of the muscle contraction will be partially sent to the cornea, where it may be detected 602 by the resistive strain sensor 106. The resistance of the sensor may modulate a fluctuating frequency 604 produced by frequency generator 118 which may be detected 606 by strain and eye movement sensor 130 in the coil 110 of the eye gear via the back-scattering signal. Based on this detected signal, the strain may be determined 608 by signal processor 120 and reported by wireless communication system 122. For example, the signal 500 of FIG. 5 is varied between a high switching frequency and a low switching frequency, with one of the frequency modes (e.g., either high or low depending on embodiment) indicating the presence of a muscle contraction strain.

It should be appreciated that in accordance with the principles disclosed herein, processing can be done at the lens level or cloud level. For example, some embodiments may include an embedded algorithm (e.g., within signal processing and interpretation circuitry 120) that may monitor for an anomaly to look for/diagnose e.g., wrong dilations during REM sleep and then send a signal using wireless communication circuitry 122 to the user's cell phone (e.g., remote system 200) to wake the user because he/she is going to enter the wrong stage of sleep (e.g., for users with PTSD). The disclosed lens system may include wireless communication circuitry 122 (Bluetooth, WiFi, Zigbee, cellular) that sends the lens data to a cloud-based network or system phone (e.g., remote system 200) where it can be processed and analyzed for anomalies, etc.

The data sent to the cloud based network or system could be used by a physician/nurse who can diagnose an anomaly and call the patient if there is bad pupil dilation. In addition, or alternatively, the cloud based network or system could send an action to the user's home which e.g., triggers a light switch (via the “Internet of Things”—IoTs) to wake up the user and to avoid bad REM sleep. These are just some example uses of the lens and system data disclosed herein.

The N.I.C.E. lens and system can be used in both animal studies and human subject studies. For example, an animal study may include simultaneously measuring multiple peripheral sympathetic nervous system (SNS) activity metrics and central norephinephrine (NE) activity. This could be an exploratory study to see which peripheral metrics best correlate with central NE activity in the locus coeruleus (LC). Moreover, the disclosed lens and system can be used to determine, among other things, (1) which peripheral metrics best correlate with central NE activity in the locus coeruleus (LC), (2) which peripheral measures best correlate with central NE activity in the LC, (3) which peripheral activation metrics best correlate with the hyperarousal and intrusive symptoms in those with PTSD, (4) which behavioral and medical (e.g. drug) interventions best lower SNS activity in those with prolonged SNS hyperactivity after trauma, (5) if SNS-lowering interventions are put in place or sleep is prevented until SNS hyperactivity is reversed, does that protect against developing the symptoms of PTSD, and (6) if sleep is allowed, but only REM sleep and TR sleep is prevented when the SNS is too high (e.g. the storms of SNS activity during sleep are exaggerated) is that enough to prevent PTSD from developing.

An example beneficiary from the disclosed N.I.C.E. lens system will be warfighters and veterans facing PTSD or TBI. The disclosed principles can measure pupil diameter and eye movement to help resolve SNS activity, especially during REM sleep. The disclosed system can provide a “green light” to users whose sympathetic readouts indicate that they are ready for adaptive sleep. However, if they are not yet ready, yet are driven to sleep, the disclosed sensor could provide an alarm-type awakening signal when it senses that the user is going into REM sleep and it would do this for the purpose of preventing maladaptive REM. Another impact is that patients who are not yet in the sleep safe zone can indicate SNS calming task that uses the disclosed metrics to indicate when they have succeeded in calming their SNS. The EEG signal helps detect REM and possible hemisphere asymmetries.

Moreover, the disclosed system provides a way to detect REM sleep and study theta power changes during sleep after trauma that track PTSD in susceptible people and help explore EEG frequency cross-coupling differences in those exposed to trauma. Another outcome from the disclosed N.I.C.E. lens system is that the military warfighter can use the tool as a device to determine mission readiness. The disclosed N.I.C.E. lens system can advance the field of sleep medicine by offering the first device to measure REM sleep and circadian rhythms. Furthermore, the disclosed lens will remove the need for many obtrusive, wired sensors used in sleep studies. Psychologists can better assess patients and their emotions with the N.I.C.E. lens system.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A system comprising:
a contact lens comprising at least one sensor; and
an eye gear package comprising:
an interface circuit configured to communicatively couple the eye gear package with the contact lens;
a power source configured to power the eye gear package and the contact lens; and
a processor configured to process data generated by the at least one sensor.

2. The system of claim 1, wherein the at least one sensor comprises a strain sensor.

3. The system of claim 2, wherein:
the contact lens further comprises an oscillation circuit;
the strain sensor is configured to modulate an output of the oscillation circuit based on detected strain;
the eye gear package further comprises strain detection circuitry configured to detect the modulated output of the oscillation circuit; and
the processor is configured to determine eye strain based on an output of the strain detection circuitry.

4. The system of claim 1, wherein the at least one sensor comprises a first coil.

5. The system of claim 4, wherein the interface circuit comprises a second coil.

6. The system of claim 5, wherein:
the eye gear package further comprises eye movement detection circuitry configured to detect a power fluctuation in power sent to the contact lens by the interface circuit caused by a change in alignment between the first coil and the second coil; and
the processor is configured to determine eye movement based on an output of the eye movement detection circuitry.

7. The system of claim 1, wherein the contact lens comprises a flexible application-specific integrated circuit comprising the at least one sensor.

8. The system of claim 1, wherein the eye gear package comprises a flexible application-specific integrated circuit comprising the interface circuit, the power source, and the processor.

9. The system of claim 1, wherein the eye gear package comprises at least one eye gear sensor.

10. The system of claim 9, wherein the at least one eye gear sensor comprises a heart rate sensor, an electroencephalogram sensor, an electrooculogram sensor, a skin impedance sensor, or a combination thereof.

11. The system of claim 1, wherein the eye gear package comprises a wireless communication system coupled to the processor and configured to transmit an output of the processor.

12. A method comprising:
powering, with a power source of an eye gear package, a contact lens comprising at least one sensor;
detecting, with the at least one sensor, an eye condition;
receiving, with an interface circuit of the eye gear package, data associated with the eye condition from the contact lens; and
processing, with a processor, the data associated with the eye condition.

13. The method of claim 12, wherein:
the at least one sensor comprises a strain sensor;
detecting the eye condition comprises modulating, with the strain sensor, an output of an oscillation circuit of the contact lens based on detected strain;
the method further comprises detecting, with strain detection circuitry of the eye gear package, the modulated output of the oscillation circuit; and
the processing by the processor comprises determining eye strain based on an output of the strain detection circuitry.

14. The method of claim 12, wherein:
the at least one sensor comprises a first coil;
the interface circuit comprises a second coil;
the method further comprises detecting, with eye movement detection circuitry of the eye gear package, a power fluctuation in power sent to the contact lens by the interface circuit caused by a change in alignment between the first coil and the second coil; and
the processing by the processor comprises determining eye movement based on an output of the eye movement detection circuitry.

15. The method of claim 12, further comprising sensing data with at least one eye gear sensor of the eye gear package.

16. The method of claim 15, wherein the data detected with the at least one eye gear sensor comprises heart rate data, electroencephalogram data, electrooculogram data, skin impedance data, or a combination thereof.

17. The method of claim 12, further comprising transmitting, with a wireless communication system coupled to the processor, an output of the processor.
